(19) United States
(12) Patent Application Publication
(10) Pub. No.: US 2020/0268283 A1
(43) Pub. Date: Aug. 27, 2020

(54) TISSUE THICKNESS MEASURING DEVICE

(71) Applicant: Covidien LP, Mansfield, MA (US)

(72) Inventors: Yogesh Kishor Vikharankar, Hyderabad (IN); Jeevan Maddur Shankar Setty, Bangalore (IN)

(21) Appl. No.: 16/786,212

(22) Filed: Feb. 10, 2020

Related U.S. Application Data
(60) Provisional application No. 62/810,973, filed on Feb. 27, 2019.

(51) Int. Cl. A61B 5/107

(52) U.S. Cl. CPC A61B 5/1075; A61B 2560/04

(57) ABSTRACT

A tissue thickness measuring device includes an elongate member defining a first lumen therethrough, an inner shaft slidably received in the first lumen, a tool assembly, and an actuation rod dimensioned to be received through the inner shaft. In particular, the tool assembly includes a neck portion and first and second jaw members. The neck portion is operatively supported on the inner shaft such that the tool assembly is transitionable between a retracted state and an extended state. The first jaw member is fixedly secured with the neck portion. The second jaw member is movable relative to the first jaw member between an approximated position and a spaced apart position. The actuation rod is configured to be selectively coupled with the second jaw member. Rotation of the actuation rod transitions the second jaw member between the approximated and spaced apart positions.

### CROSS-REFERENCE TO RELATED APPLICATION

This application claims the benefit of and priority to U.S. Provisional Patent Application No. 62/810,973 filed Feb. 27, 2019, the entire disclosure of which is incorporated by reference herein.

### BACKGROUND

### Technical Field

The disclosure relates to surgical devices, and, more particularly, to a tissue thickness measuring device.

### Background of Related Art

Endoscopic surgical procedures are performed through a small incision (usually about 5 mm to about 15 mm) or through narrow endoscopic tubes (cannulas) inserted through a small entrance wound in the skin. Endoscopic surgical procedures are often preferred over traditional open surgical procedures since a smaller incision tends to reduce the post-operative recovery time and complications.

In endoscopic surgical procedures utilizing energy based devices, the selection of proper voltage depends on the tissue thickness, and in endoscopic surgical procedures utilizing mechanical devices, size selection of staples and clips also depends on the tissue thickness. Often times, the clinician makes the thickness determination based upon visual observations of the tissue on a monitor or, if possible, the clinician uses the hands to feel the thickness of the tissue and estimates the tissue thickness.

### SUMMARY

In accordance with an embodiment of the disclosure, a tissue thickness measuring device includes an elongate member defining a first lumen therethrough, an inner shaft slidably received in the first lumen, a tool assembly, and an actuation rod dimensioned to be received through the inner shaft. In particular, the tool assembly includes a neck portion and first and second jaw members. The neck portion is operatively supported on the inner shaft such that the tool assembly is transitionable between a retracted state and an extended state. The first jaw member is fixedly secured with the neck portion. The second jaw member is movable relative to the first jaw member between an approximated position and a spaced apart position. The actuation rod is configured to be selectively coupled with the second jaw member. Rotation of the actuation rod transitions the second jaw member between the approximated and spaced apart positions.

In an embodiment, the first and second jaw members may be orthogonal to the elongate member when the tool assembly is in the extended state.

In another embodiment, the inner shaft may further include an annular member dimensioned to be slidably associated with the inner shaft. The annular member may be coupled with the neck portion of the tool assembly.

In yet another embodiment, the neck portion of the tool assembly may be pivotably coupled to the annular member.

In still yet another embodiment, the inner shaft may further include a rod coupled with the annular member for concomitant axial displacement therewith.

In an embodiment, the inner shaft may define a second lumen therethrough, and the neck portion may define a third lumen dimensioned to threadably receive the actuation rod. The third lumen may be in communication with the second lumen of the inner shaft when the tool assembly is in the extended state.

In another embodiment, the second jaw member may define a bore dimensioned to operatively engage the actuation rod.

In still another embodiment, the second jaw member may include a finger slidably supported with the first jaw member.

In yet another embodiment, the second jaw member may be configured to threadably engage the actuation rod such that rotation of the actuation rod causes axial displacement of the second jaw member.

In still yet another embodiment, the elongate member may include a mount, and the tool assembly may include an anchoring support configured to be secured to the mount to fix an orientation of the tool assembly.

In yet another embodiment, the mount may define a slot dimensioned to frictionally secure the anchoring support therein.

In still yet another embodiment, the first and second jaw members may include respective planar surfaces configured to receive tissue.

In accordance with another embodiment of the disclosure, a surgical device includes a shaft defining a lumen, a tool assembly selectably rotatable relative to the shaft, and an actuation rod. The tool assembly includes first and second jaw members. The first jaw member is movable relative to the second jaw member. The actuation rod is configured to selectively engage the first jaw member to move the first jaw member relative to the second jaw member to receive tissue between the first and second jaw members.

In an embodiment, the actuation rod may include indicia to indicate axial displacement thereof relative to the shaft.

In another embodiment, the tool assembly may be pivotable relative to the shaft between a first position in which the tool assembly is parallel to a longitudinal axis defined by the shaft, and a second position in which the tool assembly is orthogonal to the longitudinal axis.

In yet another embodiment, the shaft may further include an annular member slidably received in the shaft, and a rod coupled with the annular member for concomitant axial displacement therewith.

In still yet another embodiment, axial displacement of the annular member may transition the tool assembly between a first position in which the surgical device has a first cross-sectional dimension, and a second position in which the tool assembly has a second cross-sectional dimension greater the first cross-sectional dimension.

In accordance with another embodiment of the disclosure, a tissue thickness measuring device includes a tool assembly and an actuation rod. The tool assembly is transitionable between a retracted state and an extended state. The tool assembly includes a neck portion, a first jaw member fixedly secured with the neck portion, and a second jaw member movable relative to the first jaw member between an approximated position and a spaced apart position. The actuation rod is configured to be selectively coupled with the second jaw member. Rotation of the actuation rod transitions the second jaw member between the approximated and spaced apart positions.

### BRIEF DESCRIPTION OF THE DRAWINGS

The above and other aspects, features, and advantages of the disclosure will become more apparent in light of the following detailed description when taken in conjunction with the accompanying drawings in which:

FIG. 1 is a partial side cross-sectional view of a tissue thickness measuring device in accordance with an embodiment of the disclosure;

FIG. 2 is a side view of the tissue thickness measuring device of FIG. 1 illustrating a tool portion of the tissue thickness measuring device in an extended state;

FIG. 3 is a partial side cross-sectional view of the tissue thickness measuring device of FIG. 1 illustrating transition of the tool portion between a retracted state and the extended state;

FIG. 4 is a partial side view of the tissue thickness measuring device of FIG. 1 illustrating jaw members of the tool portion in a spaced apart position; and

FIG. 5 is a partial side view of an actuation rod of the tissue thickness measuring device of FIG. 1.

### DETAILED DESCRIPTION

Embodiments of the disclosure will now be described in detail with reference to the drawings, in which like reference numerals designate identical or corresponding elements in each of the several views. As used herein, the term “distal,” as is conventional, will refer to that portion of the instrument, apparatus, device or component thereof which is farther from the user while, the term “proximal,” will refer to that portion of the instrument, apparatus, device or component thereof which is closer to the user. In the following description, well-known functions or constructions are not described in detail to avoid obscuring the disclosure in unnecessary detail.

Various embodiments of the tissue thickness measuring device disclosed herein may be employed in endoscopic, laparoscopic, open surgical procedures, and interventional and intraluminal procedures. In addition, the tissue thickness measuring device of the disclosure may be utilized for post-operative monitoring, diagnostics and combinations thereof. In addition, various embodiments of the tissue thickness measuring device of the disclosure may be used with devices inserted in a patient to provide visualization of the target site. These devices may be introduced into the patient through natural orifices such as, e.g., vagina and/or anus, or via a trocar or a cannula, to provide images of the surgical site or anatomic location such as the lungs, liver, stomach, gall bladder, urinary tract, reproductive tract, and intestinal tissue.

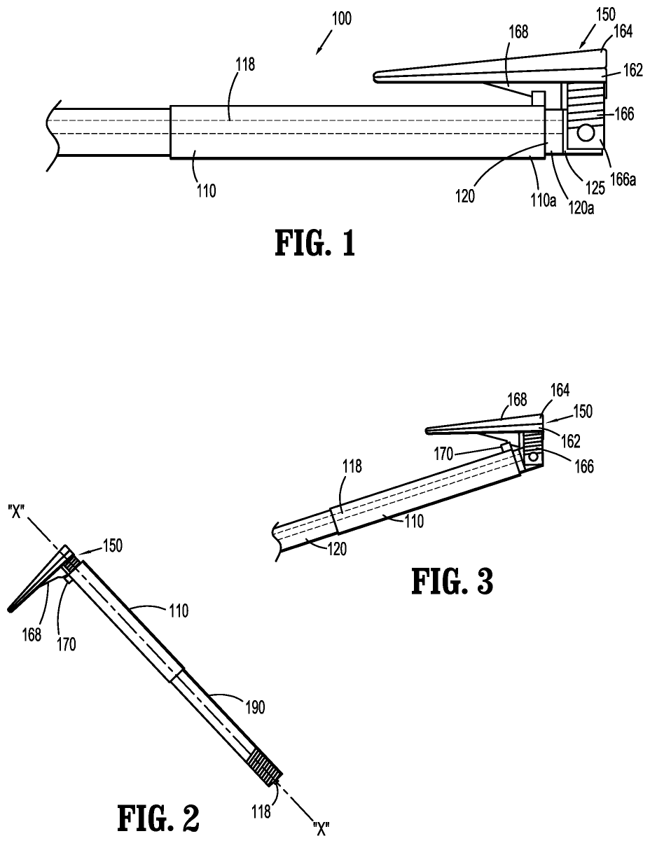

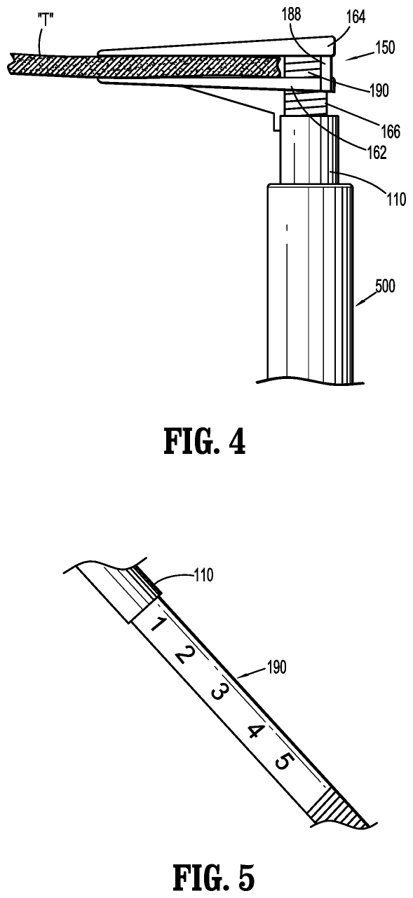

With reference now to FIG. 1, a tissue thickness measuring device is generally shown as a tissue thickness measuring device 100 configured to be received through a small opening such an orifice or an incision. Alternatively, the tissue thickness measuring device 100 may be received through, e.g., a 15 mm cannula 500 (FIG. 4). The tissue thickness measuring device 100 includes an elongate member 110, an inner shaft 120 dimensioned to be received through the elongate member 110, a tool assembly 150 operatively coupled to a distal end 120a of the inner shaft 120, and an actuation rod 190 configured to operatively engage the tool assembly 150. The tissue thickness measuring device 100 is transitionable between a retracted state (FIG. 1) in which the tissue thickness measuring device 100 has a cross-sectional dimension to facilitate insertion of the tool assembly 150 through a small incision or an opening of a trocar or the cannula 500, and an extended state (FIG. 2) in which the tissue thickness measuring device 100 is configured to measure thickness of tissue “T” (FIG. 4). The tool assembly 150 includes jaw members 162, 164. In particular, the jaw members 162, 164 may be parallel with a longitudinal axis “X-X” (FIG. 2) defined by the elongate member 110 in the retracted state, and the jaw members 162, 164 may be orthogonal to the longitudinal axis “X-X” of the elongate member 110 in the extended state.

With reference to FIGS. 2 and 3, the elongate member 110 defines a lumen (not shown) dimensioned to slidably receive the inner shaft 120. The inner shaft 120 also defines a lumen (not shown) dimensioned to receive at least a portion of the neck portion 166. In addition, a rod 118 extends through the elongate member 110 and is operatively coupled with the inner shaft 120 and an annular member 125 slidably supported on the inner shaft 120. In particular, the neck portion 166 of the tool assembly 150 is pivotably coupled to the annular member 125. The tool assembly 150 includes an anchoring support 168 configured to engage a mount 170 at a distal end 110a of the elongate member 110. In particular, the mount 170 may define a slit (not shown) dimensioned to frictionally engage the anchoring support 168 therein. Under such a configuration, when the rod 118 is advanced distally, the inner shaft 120 is displaced distally and the annular member 125 advances the neck portion 166 distally relative to the inner shaft 120, which causes the anchoring support 168 of the tool assembly 150 to disengage from the mount 170. In this manner, the neck portion 166 of the tool assembly 150 is free to pivot relative to the annular member 125. In particular, the neck portion 166 may be pivoted to place a proximal portion 166a of the neck portion 166 within the lumen of the inner shaft 120. The rod 118 may be moved proximally to, e.g., frictionally, secure the anchoring support 168 with the mount 170. Under such a configuration, the neck portion 166 is aligned with the longitudinal axis “X-X” of the elongate member 110. In addition, when the neck portion 166 is aligned with the elongate member 110, a lumen (not shown) defined by the neck portion 166 is in communication with the lumen of the inner shaft 120. In particular, an inner wall of the neck portion 166 defining the lumen includes threads configured to threadably engage the actuation rod 190.

In an embodiment, the tool assembly 150 may include a biasing member such as, e.g., a torsion spring, operatively coupled with the rod 118. The biasing member may be supported about a pivot, about which, the tool assembly 150 pivots during transition between the retracted and extended states. Under such a configuration, the tissue thickness measuring device 100 may be transitioned from the retracted state to the extended state through axial displacement of the rod 118 which provides biasing force to the tool assembly 150 to pivot to the extended state. In addition, the rod 118 may include a toggle mechanism such that through axial displacement of the rod 118, the tool assembly 150 is freed from the biasing force, thereby transitioning the tool assembly 150 back to the retracted state.

With particular reference to FIG. 4, when the neck portion 166 of the tool assembly 150 is received in the inner shaft 120 (FIG. 3), the lumen of the neck portion 166 is in communication with the lumen of the inner shaft 120 such that the actuation rod 190 is threadably received through the lumen of the neck portion 166. In particular, the anchoring support 168 disposed on the jaw member 162 engages the mount 170 on the elongate member 110 such that when the actuation rod 190 threadably engages the inner wall of the neck portion 166, the jaw member 162 is stationary relative to the elongate member 110.

With continued reference to FIG. 4, the jaw member 162 is fixedly secured with the neck portion 166, and the jaw member 164 is configured for movement relative to the jaw member 162 between an approximated position (FIG. 2) and a spaced apart position (FIG. 4). The jaw member 164 includes a finger 188 movably coupled with the jaw member 162. In particular, the jaw member 164 defines a bore (not shown) dimensioned to engage the actuation rod 190. When the jaw member 164 is spaced part from the jaw member 162, tissue “T” may be placed between jaw members 162, 164 to be measured. The jaw members 162, 164 may include planar surfaces (not shown) configured to engage tissue “T”.

The finger 188 is movably coupled with the jaw member 162 such that when the actuation rod 190 is rotatably received through the neck portion 166, the actuation rod 190 threadably engages the jaw member 164 and transitions the jaw member 164 between the approximated and spaced apart positions.

With reference to FIG. 5, the actuation rod 190 may include indicia to indicate the amount of axial displacement of the actuation rod 190, which, in turn, enables the clinician to measure a distance between the jaw members 162, 164, thereby measuring the thickness of tissue “T” (FIG. 4) disposed therebetween.

In use, an initial incision may be made by, e.g., a scalpel. Optionally, a trocar or an access port such as the cannula 500 (FIG. 4) may be utilized. The cannula 500 may be inserted through the incision and directed to a surgical site. The tissue thickness measuring device 100 in the retracted state may be inserted through the incision via the cannula 500. At this time, the tool assembly 150 extending through the cannula 500 may be transitioned to the extended state by advancing the rod 118 such that the jaw members 162, 164 are substantially orthogonal to the longitudinal axis “X-X” of the elongate member 110. At this time, the lumen of the neck portion 166 is in communication with the lumen of the inner shaft 120 to receive the actuation rod 190. Thereafter, the jaw members 162, 164 are transitioned to the spaced apart position through rotation of the actuation rod 190 such that the jaw members 162, 164 define a gap therebetween to receive tissue “T” to be measured. The clinician may rotatably adjust the actuation rod 190 to gauge the thickness of the tissue “T”. The clinician may obtain the tissue thickness by measuring axial displacement of the actuation rod 190 relative to the elongate member 110. This can be done by reading the indicia on the actuation rod 190. In this manner, the clinician need not make thickness determination based upon visual observations of the tissue on a monitor or through the use of the hands to feel the thickness of the tissue and estimate the tissue thickness. After measuring the tissue thickness, the clinician may perform a desired surgical procedure.

It will be understood that various modifications and changes in form and detail may be made to the embodiments of the disclosure without departing from the spirit and scope of the invention. For example, the actuation rod 190 may define a lumen dimensioned to receive a surgical instrument therethrough. Therefore, the above description should not be construed as limiting the invention but merely as exemplifications of preferred embodiments thereof. Those skilled in the art will envision other modifications within the scope and spirit of the invention as defined by the claims appended hereto. Having thus described the invention with the details and particularity required by the patent laws, what is claimed and desired protected is set forth in the appended claims.

What is claimed is:

1. A tissue thickness measuring device comprising:
an elongate member defining a first lumen therethrough;
an inner shaft slidably received in the first lumen;
a tool assembly including:
a neck portion operatively supported on the inner shaft such that the tool assembly is transitionable between a retracted state and an extended state;
a first jaw member fixedly secured with the neck portion; and
a second jaw member movable relative to the first jaw member between an approximated position and a spaced apart position; and
an actuation rod dimensioned to be received through the inner shaft and configured to be selectively coupled with the second jaw member, wherein rotation of the actuation rod transitions the second jaw member between the approximated and spaced apart positions.

2. The tissue thickness measuring device according to claim 1, wherein the first and second jaw members are orthogonal to the elongate member when the tool assembly is in the extended state.

3. The tissue thickness measuring device according to claim 1, wherein the inner shaft further includes an annular member dimensioned to be slidably associated with the inner shaft, the annular member coupled with the neck portion of the tool assembly.

4. The tissue thickness measuring device according to claim 3, wherein the neck portion of the tool assembly is pivotably coupled to the annular member.

5. The tissue thickness measuring device according to claim 3, wherein the inner shaft further includes a rod coupled with the annular member for concomitant axial displacement therewith.

6. The tissue thickness measuring device according to claim 5, wherein the inner shaft defines a second lumen therethrough, and the neck portion defines a third lumen dimensioned to threadably receive the actuation rod, wherein the third lumen is in communication with the second lumen of the inner shaft when the tool assembly is in the extended state.

7. The tissue thickness measuring device according to claim 6, wherein the second jaw member defines a bore dimensioned to operatively engage the actuation rod.

8. The tissue thickness measuring device according to claim 6, wherein the second jaw member includes a finger slidably supported with the first jaw member.

9. The tissue thickness measuring device according to claim 8, wherein the second jaw member is configured to threadably engage the actuation rod such that rotation of the actuation rod causes axial displacement of the second jaw member.

10. The tissue thickness measuring device according to claim 1, wherein the elongate member includes a mount, and the tool assembly includes an anchoring support configured to be secured to the mount to fix an orientation of the tool assembly.

11. The tissue thickness measuring device according to claim 10, wherein the mount defines a slot dimensioned to frictionally secure the anchoring support therein.

12. The tissue thickness measuring device according to claim 1, wherein the first and second jaw members include respective planar surfaces configured to receive tissue therebetween.

13. A surgical device comprising:
a shaft defining a lumen;
a tool assembly selectably rotatable relative to the shaft, the tool assembly including first and second jaw members, the first jaw member movable relative to the second jaw member; and
an actuation rod configured to selectively engage the first jaw member to move the first jaw member relative to the second jaw member to receive tissue between the first and second jaw members.

14. The surgical device according to claim 13, wherein the actuation rod includes indicia to indicate axial displacement thereof relative to the shaft.

15. The surgical device according to claim 13, wherein the tool assembly is pivotable relative to the shaft between a first position in which the tool assembly is parallel to a longitudinal axis defined by the shaft, and a second position in which the tool assembly is orthogonal to the longitudinal axis.

16. The surgical device according to claim 13, wherein the shaft further includes an annular member slidably received in the shaft, and a rod coupled with the annular member for concomitant axial displacement therewith.

17. The surgical device according to claim 16, wherein axial displacement of the annular member transitions the tool assembly between a first position in which the surgical device has a first cross-sectional dimension, and a second position in which the tool assembly has a second cross-sectional dimension greater than the first cross-sectional dimension.

18. A tissue thickness measuring device comprising:
a tool assembly transitionable between a retracted state and an extended state, the tool assembly including:
a neck portion;
a first jaw member fixedly secured with the neck portion; and
a second jaw member movable relative to the first jaw member between an approximated position and a spaced apart position; and
an actuation rod configured to be selectively coupled with the second jaw member, wherein rotation of the actuation rod transitions the second jaw member between the approximated and spaced apart positions.

19. The tissue thickness measuring device according to claim 18, further comprising a shaft including an annular member dimensioned to be slidably associated with the shaft, the annular member coupled with the neck portion of the tool assembly such that the tool assembly is transitionable between the retracted and extended states.

20. The tissue thickness measuring device according to claim 19, wherein the neck portion of the tool assembly is pivotably coupled to the annular member.
